(19) United States
(12) Patent Application Publication
(10) Pub. No.: US 2017/0014464 A1
(43) Pub. Date: Jan. 19, 2017

(54) HERBAL COMPOSITION FOR BODY TREATMENT

(71) Applicant: FARMA-BLANCO, LLC, Denver, CO (US)

(72) Inventor: Fernando Blanco Rodriguez, Morelia Michoacan (MX)

(21) Appl. No.: 15/213,046

(22) Filed: Jul. 18, 2016

Related U.S. Application Data
(60) Provisional application No. 62/194,011, filed on Jul. 17, 2015.

(51) Int. Cl. A61K 36/77; A61K 36/48

(52) U.S. Cl. CPC A61K 36/77; A61K 36/48; A61K 2236/15; A61K 2236/37; A61K 2236/331; A61K 2236/53; A61K 2236/31

(57) ABSTRACT

The present invention discloses an herbal composition comprising a therapeutic effective amount of Cascalote (Caesalpinia coriaria) and a therapeutic effective amount of Ruda (Ruta graveolens). In another implementation, the herbal composition can also comprise of an effective amount of at least one pharmaceutically acceptable excipient for application to the body, including skin and tissue. The disclosed invention also includes a method for making the herbal composition and a method of application of the herbal composition.

### CROSS REFERENCE TO RELATED APPLICATIONS

The present application claims priority to pending U.S. Provisional Patent Application Ser. No. 62/194,011, entitled “Herbal Composition for Treatment of Skin and Tissue,” filed on Jul. 17, 2015, all of which is specifically incorporated by reference for all it discloses and teaches.

### BACKGROUND

Skin and tissue in the body become wrinkled, stretched, discolored, damaged, and loosened for a variety of reasons. For example, as a person ages, their skin wrinkles (e.g., around the eyes or near the mouth). Wrinkles can also be a result of sun exposure, in addition to aging and stress. Sun exposure can also cause discoloration of skin or sunspots. In another example, when a person gains and loses weight, the weight loss can result in stretch marks or striae disease.

In yet other examples, tissue stretches (e.g., vaginal tissue). For example, during a vaginal childbirth, vaginal tissues can weaken. Vaginal tissues can also loosen as a result of age, infection, hysterectomy, or other causes. This can negatively interfere with the sexual response. In yet other examples, trauma or surgery can have damaging effects on skin or tissue, including scars.

As a person ages and/or loses tightness in tissue and skin or suffers damage, it becomes desirable to reverse these effects. To address these bodily conditions, many people use lotions, gels, creams, ointments, herbal compositions or other treatments to shrink, tighten, firm, and treat skin or tissue. Often, these treatments are not effective and/or the effects of the treatments are not long-term.

### SUMMARY

The disclosed compositions include herbal compositions for application to a user's body to prevent, treat, and/or cure skin, tissue, and other bodily conditions. Applications include treatment of wrinkles, skin discoloration, and other skin or tissue damage or imperfections. In other implementations, the herbal compositions can be used to treat other bodily conditions, such as dental conditions or gastroenterological conditions. In one implementation, a disclosed composition provides for an herbal composition comprising a therapeutic effective amount of Cascalote (Caesalpinia coriaria) and Ruda (Ruta graveolens). Also described herein are methods for making herbal compositions and methods of using herbal compositions.

### BRIEF DESCRIPTION OF THE DRAWINGS

A further understanding of the nature and advantages of the present composition may be realized by reference to the FIGURE, which is described in the remaining portion of the Specification.

FIG. 1 illustrates example operations for manufacturing the herbal composition.

### DETAILED DESCRIPTIONS

Implementations of the herbal compositions as disclosed herein provide for multi-purpose body treatment, comprising extracts of Cascalote (Caesalpinia coriaria) and Ruda (Ruta graveolens), to reduce, diminish, remove, treat, and prevent skin roughness and dryness, acne, wrinkles, scars, and other skin imperfections, heal cuts and wounds, reduce or eliminate skin discoloration, shrink skin and tissue, increase skin firmness and elasticity, and delay aging, in addition to other uses and treatments for other skin and tissue implementations. The herbal compositions can be used to treat, repairs, and strengthen hair, nails and cuticles. The herbal compositions can also be used to treat dental conditions (e.g., toothache), gastroenterological conditions (i.e., acid reflux and ulcers), and other bodily ailments.

Specifically, the disclosed compositions include herbal compositions comprising a therapeutic effective amount of Cascalote and a therapeutic effective amount of Ruda. The compositions can be administered alone, or they can be mixed with at least one pharmaceutically or cosmetically acceptable carrier or diluent. For example, a composition may comprise of an herbal composition comprising a therapeutic effective amount of Cascalote, a therapeutic effective amount of Ruda, and water. Also described are processes for making and using herbal compositions and processes for using the herbal compositions.

The safe and therapeutic effective amounts of Cascalote and Ruda can be measured by the amount of plant extract of each respective plant. Cascalote (Caesalpinia coriaria) is a leguminous tree or large shrub native to the Caribbean, Mexico, Central America, and northern South America. Tannins can be extracted and corilagin molecules can be isolated from Cascalote.

Ruda (Ruta graveolens) is native to the Balkan Peninsula and grown throughout the world as an ornamental plant, and for other uses. Ruda comprises a number of chemicals, such as furanoacridones, two acridone alkaloids (arborinine and evoxanthine), coumarins and limonoids. Cell cultures of Ruda can produce coumarins umbelliferone, scopoletin, psoralen, xanthotoxin, isopimpinellin, rutamarin and rutacultin (6,7-dimethoxy-3-(1,1-dimethylallyl)coumarin), and the alkaloids skimmianine, kokusaginine, 6-methoxydictamnine and edulinine (1-methyl-4-methoxy-3-[2,3-dihydroxy-3-methylbutyl]-2-quinolone). The ethyl acetate extract of Ruda leaves yields two furanocoumarins, one quinoline alkaloid and four quinolone alkaloids. The chloroform extracts of the root, stem and leaf shows the isolation of furanocoumarin chalepensin. The essential oil of Ruda contains two main constituents undecan-2-one and nonan-2-one.

In exemplary implementations comprising at least one pharmaceutically acceptable excipient (or carrier), the pharmaceutically acceptable excipient may include one or more of a plant (e.g., Arnica), an antioxidant, a thickening agent, a vaginal conditioning agent, a vasodillating agent, preservative, lubricating agent, and water (e.g., boiled water, distilled water, filtered water, etc.). The pharmaceutically acceptable excipient may be a natural or synthetic substance formulated with the active ingredients of Cascalote and Ruda. The pharmaceutically acceptable excipient may be included for the purpose of long-term stabilization, bulking up solid formulations that contain potent active ingredients (e.g., bulking agents, fillers, or diluents), or to confer a therapeutic enhancement on the active ingredient in the final dosage form, such as facilitating absorption, reducing viscosity, or enhancing solubility.

The pharmaceutically acceptable excipient may be useful in the manufacturing process, to aid in the handling of the active substance concerned such as by facilitating powder flowability or non-stick properties, in addition to aiding in vitro stability such as prevention of denaturation or aggregation over the expected shelf life. The selection of appropriate pharmaceutically acceptable excipients may depend upon the route of administration and the dosage form, as well as the active ingredient and other factors. There may be other pharmaceutically acceptable excipients depending on the intended use and treatment.

The disclosed compositions include a variety of formulations in which the amount and selection of a pharmaceutically acceptable excipient or carrier may vary depending on an intended site of treatment (e.g., an arm, a tooth, a stomach, etc.) and intended method of delivery (e.g., topical treatment, ingestion). In some implementations, the pharmaceutically acceptable excipient may be an orally or topically acceptable carrier and/or an orally or topically acceptable diluent. For example, for application to skin on the face or hands, lotions may be desirable for delivery, whereas gels or sprays may be desirable for tooth application. The disclosed compositions may optionally include a wide variety of additional components intended to improve the overall desirability, visual appearance, and physical properties of the herbal compositions.

In some implementations, coloring agents (e.g., natural dye, synthetic dye, semi-permanent dye, direct dye) may be added to the herbal compositions. In some implementations, antioxidants, germicidal agents, preservatives, and other additives may be added to the herbal compositions. In some implementations, aromatic or fragrance may be added to the herbal compositions for taste or smell. For example, rose oil, peppermint oil, menthol, borneol, eucalyptus oil, apple flavor, lemon flavor, orange flavor or essential oils may be added to the herbal compositions.

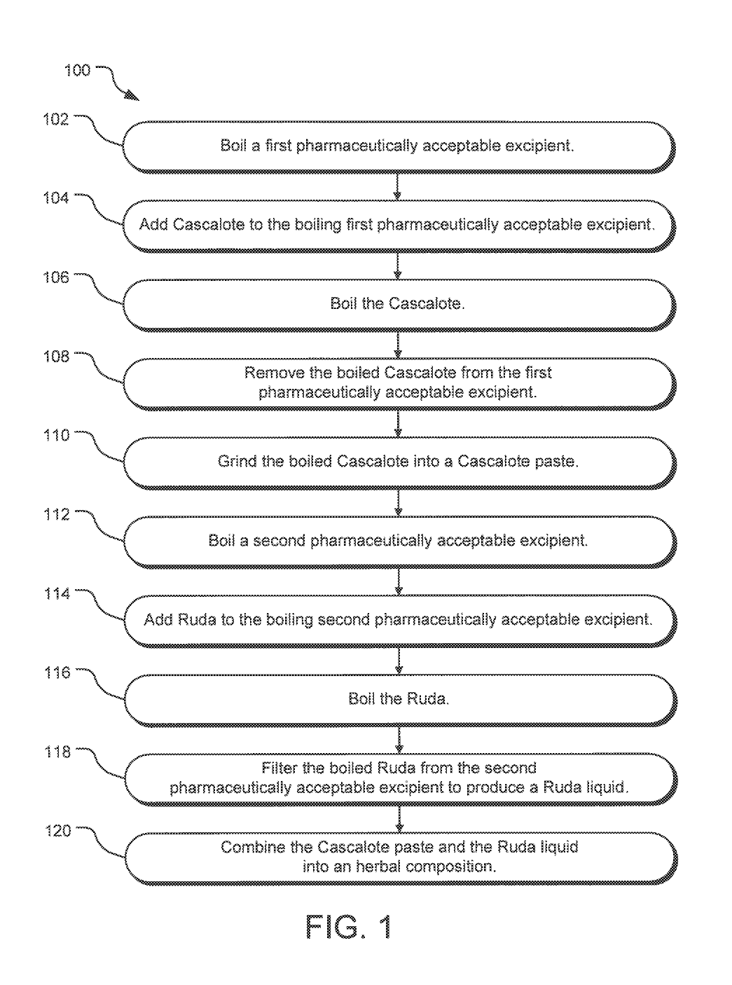

FIG. 1 shows operations 100 for manufacturing the herbal composition described herein. In an operation 102, a first pharmaceutically acceptable excipient is boiled in a first container. The pharmaceutically acceptable excipients described herein can be an antioxidant, a thickening agent, a vaginal conditioning agent, a vasodillating agent, preservative, lubricating agent, and water (e.g., boiled water, distilled water, filtered water, etc.), although other pharmaceutically acceptable excipients may be used. In this implementation, the first pharmaceutically acceptable excipient is water.

An operation 104 adds Cascalote to the boiling pharmaceutically acceptable excipient. In one implementation, the amount of Cascalote can comprise the seed of the Cascalote tree. However, in other implementations, other parts of the Cascalote tree may be used. In some implementations where the seed of the Cascalote tree is used, the therapeutic effective amount of Cascalote is made from 0.25-2 lbs of Cascalote per 1 gallon of pharmaceutically acceptable excipient. There are other contemplated concentrations.

An operation 106 boils the seeds of Cascalote in the boiling water (e.g., 30-40 minutes). However, in other implementations, the time of boiling may be less or more than 30-40 minutes. An operation 108 removes the boiled seeds of Cascalote from the boiling water. An operation 110 grinds the boiled seeds of Cascalote into a Cascalote paste. The mechanism for grinding may vary (e.g., grinder, blender, processor, etc.).

An operation 112 boils a second pharmaceutically acceptable excipient in a second container. In this implementation, the second pharmaceutically acceptable excipient is water. An operation 114 adds Ruda to the second container of boiling water. In this implementation, the amount of Ruda can include the flower and/or leaves of the Ruda plant. However, in other implementations, other selected parts of the Ruda tree may be used. In some implementation, the therapeutic effective amount of Ruda is made from 0.25-2 oz. of Ruda per 1 gallon of water. In one implementation, 2 oz. of Ruda are used with 1 gallon of water to yield a stronger mixture. In yet another implementation, there may be one-half to one gallon of water used. There are other contemplated concentrations.

An operation 116 boils the flower and/or leaves of Ruda in the second container of boiling water. An operation 118 filters the boiled flower and/or leaves of Ruda from the second container separating out a Ruda liquid. In one implementation, operation 118 strains the Ruda through a porous or perforated device to separate out any or at least the majority of any solid matter.

In other implementations, other devices or methods may be used to obtain a Ruda liquid (e.g., percolation, concentrating the plant extract to dryness on a rotatory evaporator, in a steam bath, etc.) In another implementation, a method of manufacturing the herbal composition can include extraction of the therapeutic effective amounts of the Cascalote and Ruda plants using one or more organic solvents (e.g., including n-hexane, chloroform, dichloromethane, ethyl acetate, acetone, alcohol, methanol and water).

A final operation 120 combines the Cascalote paste and the Ruda liquid into an herbal composition. In one implementation, the final operation 120 is performed when both the Cascalote paste and the Ruda liquid are hot (e.g., in a range from room temperature to 100° F.). In another implementation, the Cascalote paste and the Ruda liquid are mixed at room temperature.

In other implementations, operations 112-118 may occur before or at the same time as operations 102-110. In other implementations, one or more pharmaceutically acceptable excipients may be incorporated into the operations 101-118. For example, a third pharmaceutically acceptable excipient may be boiled alone or with the first and/or second pharmaceutically acceptable excipient.

In one implementation, the effective amount of Cascalote is in the range of 75% w/w of the total herbal composition, although the effective amount may be more or less than that given amount. In one implementation, the effective amount of Ruda plant extract is in the range of 25% w/w of the total herbal composition, although the effective amount may be more or less than that given amount. In other implementations, one or more pharmaceutically acceptable excipients or other additives may be used in operations 102 and 112. In other implementations, one or more pharmaceutically acceptable excipients or other additives may be added to the herbal composition following operation 118, which can vary in amounts of the total composition.

The disclosed herbal compositions can be used for the treatment or prevention of medical conditions and/or a method of cosmetic treatment of a human or other animal. Medical conditions which are provided by the present invention include rashes and allergic reactions, inflammations, bacterial infections and gastrointestinal problems, but are not limited thereto.

The compositions of this invention can suitably be given in dosing and are prepared by methods known in the art of pharmacy and cosmetics procedures. For example, pharmacy administration can include any mode of administration that is topically, internally, or orally acceptable, that is, each way, the effective level of the active ingredient of the invention produced without causing clinically unacceptable adverse effects.

The disclosed herbal compositions can have many routes of administration depending on the particular condition being treated and the dosage required for therapeutic, prophylactic or cosmetic efficacy. Specifically, the amounts of Cascalote, Ruda, and pharmaceutically acceptable excipient(s) and/or additives can vary depending on the intended use as well as the administration. To deliver the active ingredients topically, for example, certain carrier bases may be used (e.g., gel, alcohol, water, cream ointment, salves, lotion, liniment, tinctures, cream gel, lotion ointment, lotion cream, aerosol, lotion spray, balm rub, gel ointment, poultice, plaster, infusion, decoction). For example, a vaginal wash or ointment may be less concentrated than a topical gel or cream. In another example, a composition for acid reflux treatment may be a liquid.

The herbal compositions can be administered by a variety of methods (e.g., dermal, urogenital, rectal, etc.). For example, in a dermal administration, numerous formulations can be used to apply the herbal composition directly (e.g., cream, topical, gel, foam, lotion, ointment, mask, shower gel, wash, drops, suppositories, etc.) to skin. In a urogenital administration, an herbal composition (e.g., vaginal cream or wash) may be used. In a rectal administration, an herbal composition (e.g., a suppository) may be used. In other implementations, there may be other methods of application. Such routes of administration can include pills, water, alcohol, sugar pills, glycerin, milk sugar and cane sugar vehicles, medicated powders, medicated globules (pellets, pilules), cones, drops, tablets, gum, mouthwashes, mouth lotions, gargle solutions, toothpastes, emmenagogues, creams, paints, sprays, pastes, liniments, lotions, ointments, etc.

Cosmetic treatments that are provided by the present invention include skin and/or hair treatments and/or oral treatments, such as odor-controlling agents, facial scrubs, shaving creams and gels, eye/face creams and gels, make-up Erstferner, toner, detergent, shampoos, conditioners and breath freshener, but are not limited thereto.

In one example, a topical administration of the herbal composition includes washing the bodily area prior to administration. The herbal composition is applied to the bodily area daily. The bodily area daily can be covered. In some applications, results of repair, diminished marks, and/or healing will be observed within four to five weeks. In other implementations, depending on the condition of the bodily area and other factors, results may appear sooner or take longer. In some implementations for vaginal use, results can be observed in approximately five weeks.

In some examples for treatment of toothaches and gastritis, a liquid mixture of the herbal composition (e.g., the liquid mixture described in FIG. 1) can be further diluted into a cup of water or other pharmaceutically acceptable excipient. For a toothache, a diluted liquid mixture can be used as a mouthwash (e.g., one daily dose for up to eight days). For gastritis, a dose of a diluted liquid mixture can be ingested (e.g., one daily dose within a five-minute time period, for up to 15 days).

The disclosed herbal compositions can be stored in the refrigerator. In some implementations, the herbal compositions hold efficacy for about three months. However, the herbal compositions can also be stored at room temperature. In one implementation, the herbal composition can be stored at room temperature for as many as three days. In another implementation, the herbal composition can be stored at room temperature for as many as ten days, if the amount of Ruta is doubled during manufacturing.

The above specification, examples, and data provide a complete description of the structure and use of exemplary embodiments of the invention. Since many embodiments of the invention can be made without departing from the spirit and scope of the invention, the invention resides in the claims hereinafter appended. Furthermore, structural features of the different embodiments may be combined in yet another embodiment without departing from the recited claims.

What is claimed is:

1. An herbal composition comprising:
a therapeutic effective amount of Cascalote; and
a therapeutic effective amount of Ruda.

2. The herbal composition of claim 1, further comprising:
an effective amount of at least one pharmaceutically acceptable excipient.

3. The herbal composition of claim 1, wherein a range of therapeutic effective amount of Cascalote is made from 0.25-2 lbs. of Cascalote per one gallon of pharmaceutically acceptable excipient.

4. The herbal composition of claim 1, wherein a range of therapeutic effective amount of Ruda is made from 0.25-2 oz. of Ruda per one gallon of pharmaceutically acceptable excipient.

5. The herbal composition of claim 2, wherein the at least one pharmaceutically acceptable excipient is in a range of 90% to 98% w/w of the total composition of the herbal composition.

6. The herbal composition of claim 2, wherein the pharmaceutically acceptable excipient comprises at least one of astringent, antioxidant, vaginal conditioning agent, vasodillating agent, thickening agent, preservative, lubricating agent, colorant, and water.

7. The herbal composition of claim 2, wherein the pharmaceutically acceptable excipient may be an orally or topically acceptable carrier and/or an orally or topically acceptable diluent.

8. The herbal composition of claim 1, for the treatment of at least one of skin and tissue.

9. The herbal composition of claim 1, for the treatment of a gastroenterological condition.

10. The herbal composition of claim 1, for the treatment of a dental condition.

11. A method comprising:
boiling a first pharmaceutically acceptable excipient;
adding Cascalote to the boiling first pharmaceutically acceptable excipient;
boiling the Cascalote in the boiling first pharmaceutically acceptable excipient;
removing the boiled Cascalote from the boiling first pharmaceutically acceptable excipient;
grinding the boiled Cascalote into a Cascalote paste;
boiling a second first pharmaceutically acceptable excipient;
adding Ruda to the boiling second pharmaceutically acceptable excipient;
boiling the Ruda in the boiling second pharmaceutically acceptable excipient;
filtering the boiled Ruda from the boiling second pharmaceutically acceptable excipient;
combining the Cascalote paste and the second pharmaceutically acceptable excipient into an herbal composition.

12. The method of claim 11, wherein the first pharmaceutically acceptable excipient is water.

13. The method of claim 11, wherein the second pharmaceutically acceptable excipient is water.

14. The method of claim 11, further comprising adding a third pharmaceutically acceptable excipient to the herbal composition.

15. The method of claim 11, wherein a range of therapeutic effective amount of Cascalote is made from 0.25-2 lbs. of Cascalote per one gallon of pharmaceutically acceptable excipient.

16. The method of claim 11, wherein a range of therapeutic effective amount of Ruda is made from 0.25-2 oz. of Ruda per one gallon of pharmaceutically acceptable excipient.

17. The method of claim 11, wherein the amount of first pharmaceutically acceptable excipient and the second pharmaceutically acceptable excipient is in a range of 90% to 98% w/w of the total composition.

18. The method of claim 11, for the treatment of at least one of stretch marks, acne, wrinkles, skin discoloration, stretching of connective tissue, and scars.

19. A method comprising:
applying an herbal composition to treat a bodily condition, the herbal composition comprising a mixture of Cascalote, Ruda, and at least one pharmaceutically acceptable excipient.

20. The method of claim 18, wherein applying an herbal composition includes oral or topical administration.
